(19) United States
(12) Patent Application Publication
(10) Pub. No.: US 2021/0050076 A1
(43) Pub. Date: Feb. 18, 2021

(54) AGGREGATING VERIFIED HEALTH PROFILES IN EMERGENCY ROOMS

(71) Applicant: International Business Machines Corporation, Armonk, NY (US)

(72) Inventors: Charles Muchiri Wachira, Karatine (KE); Samuel Osebe, Nairobi (KE); William Ogallo, Nairobi (KE); Aisha Walcott, Nairobi (KE); Fiona Mugure Matu, Nairobi (KE)

(21) Appl. No.: 16/543,542

(22) Filed: Aug. 17, 2019

(51) Int. Cl. G16H 10/60; G16H 50/30

(52) U.S. Cl. CPC G16H 10/60; G16H 50/30

(57) ABSTRACT

Identify an emergency patient's potential emergency contacts and their social relationships by processing the patient's Internet footprint with a first natural language processing algorithm. Identify the emergency patient's possible health context by processing at least one of the emergency patient's Internet footprint and diagnostic data with a second natural language processing algorithm. Link at least one item of the emergency patient's possible health context to at least one linked emergency contact, who is selected from the potential emergency contacts using a sorting module that is trained to associate types of medical conditions with attributes of emergency contacts. Verify the at least one item of the emergency patient's possible health context by contacting the at least one linked emergency contact. Aggregate a verified health profile for the emergency patient based on at least one response provided by the at least one linked emergency contact.

### BACKGROUND

The present invention relates to the medical arts, and more specifically, to systems for maintenance of electronic health records.

In emergency room admissions, medical histories are helpful for diagnosis and triage of incoming patients. Prompt triage and diagnosis are especially important in emergency rooms because of the high number of incoming patients. For example, in 2016, about 146 million patients visited emergency rooms in United States hospitals. Most of those patients were conscious on arrival and were capable of delivering a medical history to the emergency room personnel. Some, however, were not conscious. Those few typically would be triaged ahead of the conscious patients, but diagnosis could be complicated by their inability to deliver a medical history.

### SUMMARY

Principles of the invention provide techniques for aggregating verified health profiles in emergency rooms. In one aspect, an exemplary method includes identifying an emergency patient's potential emergency contacts and social relationships of the potential emergency contacts to the emergency patient by processing the emergency patient's Internet footprint with a first natural language processing algorithm that is trained for social relationships; then identifying the emergency patient's possible health context by processing at least one of the emergency patient's Internet footprint and diagnostic data with a second natural language processing algorithm that is trained for medical information using a generic classifier. The exemplary method also includes linking at least one item of the emergency patient's possible health context to at least one linked emergency contact, who is selected from the potential emergency contacts using a sorting module that is trained to associate types of medical conditions with attributes of emergency contacts, and verifying the at least one item of the emergency patient's possible health context by contacting the at least one linked emergency contact, then aggregating a verified health profile for the emergency patient based on at least one response provided by the at least one linked emergency contact.

One or more embodiments of the invention or elements thereof can be implemented in the form of a computer program product including a computer readable storage medium with computer usable program code for performing or facilitating the method steps indicated. Furthermore, one or more embodiments of the invention or elements thereof can be implemented in the form of a system (or apparatus) including a memory that embodies computer executable instructions, and at least one processor that is coupled to the memory and operative by the instructions to perform or to facilitate exemplary method steps. Yet further, in another aspect, one or more embodiments of the invention or elements thereof can be implemented in the form of means for carrying out one or more of the method steps described herein; the means can include (i) hardware module(s), (ii) software module(s) stored in a tangible computer readable storage medium (or multiple such media) and implemented on a hardware processor, or (iii) a combination of (i) and (ii); any of (i)-(iii) implement the specific techniques set forth herein.

As used herein, “facilitating” an action includes performing the action, making the action easier, helping to carry the action out, or causing the action to be performed. Thus, by way of example and not limitation, instructions executing on one processor might facilitate an action carried out by instructions executing on a remote processor, by sending appropriate data or commands to cause or aid the action to be performed. For the avoidance of doubt, where an actor facilitates an action by other than performing the action, the action is nevertheless performed by some entity or combination of entities.

In view of the foregoing, techniques of the present invention can provide substantial beneficial technical effects. For example, one or more embodiments provide one or more of:

Ability to provide, to emergency room caregivers, emergency medical information that cannot be obtained directly from an emergency medical patient or from the patient's formal health records.

Ability to augment/enrich patient-specific emergency situation information for secondary use (e.g. quality assurance, outbreak surveillance, data science)

Ability to keep patients and their potential emergency contacts informed and activated in ongoing emergency situations.

Ability to augment/enrich emergency situation information for ameliorating exposure to possible litigation and arbitration challenges

These and other features and advantages of the present invention will become apparent from the following detailed description of illustrative embodiments thereof, which is to be read in connection with the accompanying drawings.

### BRIEF DESCRIPTION OF THE DRAWINGS

FIG. 1 depicts in a schematic a system for aggregating verified health profiles in an emergency room, according to an exemplary embodiment;

FIG. 2 depicts in a flowchart an algorithm that is implemented by the system of FIG. 1, according to an exemplary embodiment;

FIG. 3 depicts in a flowchart an algorithm for linking a potential emergency contact to a health context, according to an exemplary embodiment; and

FIG. 4 depicts a computer system that may be useful in implementing one or more aspects and/or elements of the invention.

### DETAILED DESCRIPTION

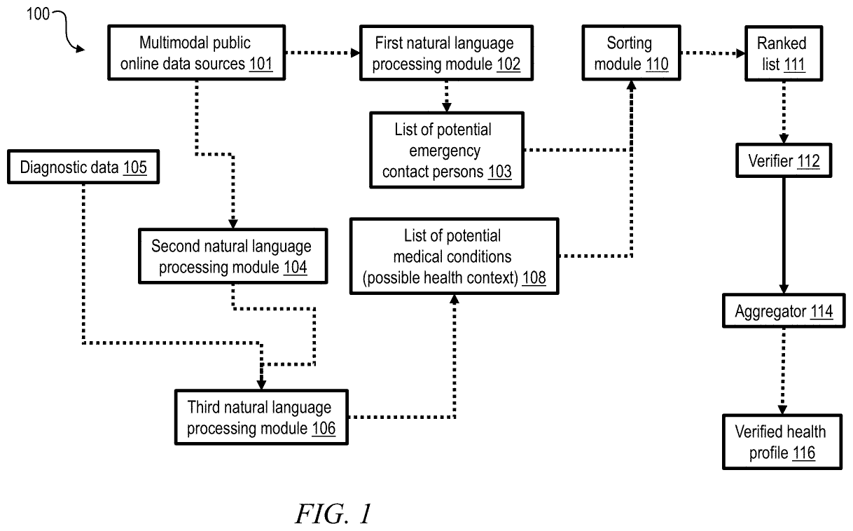

FIG. 1 depicts in a schematic a system 100 for providing a non-documented patient's health profile and context (e.g., conditions managed, medication history, potential for contraindications, allergic reactions, drug-drug reactions and food-drug reactions) that would help to inform health care providers in stabilizing and treating a case in the emergency room (ER). The system 100 includes a first natural language processing module (NLP) 102 that is trained to identify social relationships and that accesses multimodal public online data sources 101 (i.e. the patient's Internet footprint including social media data) to create a list 103 of potential emergency contact persons (PECPs) in response to a patient's identity. The first NLP identifies potential emergency contact persons using a recurrent neural network-based approach for named entity recognition, as described, for example, by Li et al. in “A Survey on Deep Learning for Named Entity Recognition” (2018). The data for training this model may comprise existing corpora from multiple sources including social media and phone text messages etc. The training corpora would be already annotated or readily annotatable with named entities (in this case person names). All the corpora used in building the model are preprocessed and tokenized. The model is trained to take sequences of word tokens (a text corpus) as input, and to generate as output a list of person names (named entities) that are mentioned in the text corpus. This output can be further characterized by cross-referencing with an individual's social network graph generated (with a priori consent of the individual) from friend lists, phone contact lists, etc.

The system 100 also includes a second (generic) natural language processing module 104 and a third (customized) NLP 106 that are trained to identify medical conditions and terminology and that access the multimodal public online data sources 101 and the patient's diagnostic data 105 (if any) (including, e.g., vital signs, first responder reports, and any other formal medical history that is available) to create a list 108 of potential medical conditions. In particular, the generic classifier 104 is trained to parse the multimodal public online data sources 101 while the customized NLP 106 is trained to parse the diagnostic data 105. The second NLP also comprises a recurrent neural network-based approach for named entity recognition. This model is specifically trained to take as input a corpus of text, and generate as output a list of clinical findings, signs, and symptoms identified from the text corpus. The generated list can be further characterized by mapping them to existing medical ontologies (e.g. SNOMED-CT).

In one or more embodiments, the customized NLP 106 is trained, for example, using domain specific data such as a drug index, a drug bank, or U.S. Food and Drug Administration (FDA) open source data. (A “drug bank” means a detailed database containing information about drugs, similar to a drug index.) The third NLP uses a named entity recognition approach to process unstructured diagnostic data and subsequently identify a list of conditions described in the diagnostic input data. This model can be trained using unstructured electronic health records that have been annotated. A matching/mapping algorithm, together with ontologies from existing standard vocabularies e.g. drug index, drug bank, FDA etc., may also be used to match any clinical findings, signs, and symptoms identified in the input to a list of potential medical conditions condition.

The system 100 also includes a sorting module 110 that generates a ranked list 111 of the potential emergency contact persons in response weighted associations between the contact persons' attributes (e.g., demography, relationship to patient, location, and interests) and the potential medical conditions. Weighted associations between attributes and medical conditions are determined using a model based on de-identified patient data and domain knowledge data repositories. The domain knowledge comprises data/information/knowledge from different knowledge sources including published literature, spontaneous reporting repositories such as FDA Adverse Event Reporting System, existing clinical care guidelines, structured product labels, and standardized medical terminologies, ontologies, and coding systems. The sorting module 110 also may adjust the ranked list 111 in response to severity of potential medical conditions as identified by the third natural language processing module 106. The clinical findings, signs, and symptoms uncovered by the second NLP and processed by the third NLP from diagnostic data input, may be cross-referenced with information from domain knowledge to determine the severity of a condition e.g. by evaluating clusters and classifications of clinical findings, signs, and symptom.

The system 100 also includes a verifier 112 that contacts at least one of the potential emergency contact persons consistent with the ranked list 111, i.e. first contacting the person at the top of the list. Upon contacting a PECP, the verifier 112 initiates an automated health condition inquiry related to the possible medical condition that is associated with that PECP in the ranked list. The system matches identified medical condition to the right PECP based on context. For example, if the patient has a diabetes-related emergency, a given PECP would be ranked higher in the ranked list while if the patient has a pregnancy-related emergency, the same PECP would be ranked lower.

Once the right PECP has been identified, the system generates a list of probing questions to which the PECP would respond to verify the accuracy of information gathered as well as request addition information about the patient. These questions and response options can be customized based on the patient, PECP, and conditions being probed. An example of this is shown below:
Hi Mary, your friend John has recently been involved in a medical emergency.
We would like you to provide us with information that would be helpful in his care.
Which of the following information is accurate about patient John?
John has diabetes [Yes, No, Don't Know]
John drinks alcohol [Yes, No, Don't Know]
John smokes [Yes, No, Don't Know]
John recently travelled to central Africa [Yes, No, Don't Know]
What additional relevant information may be useful in caring for patient John?
[Mary's response would be inserted here]

Finally, the system 100 includes a health profile aggregator 114 that aggregates a verified health profile 116 for a patient by receiving and collating responses from the patient's potential emergency contact persons (e.g., using speech-to-text technology and a third natural language processing algorithm to generate medical record entries from unstructured verbal responses to automated health conditions inquiries).

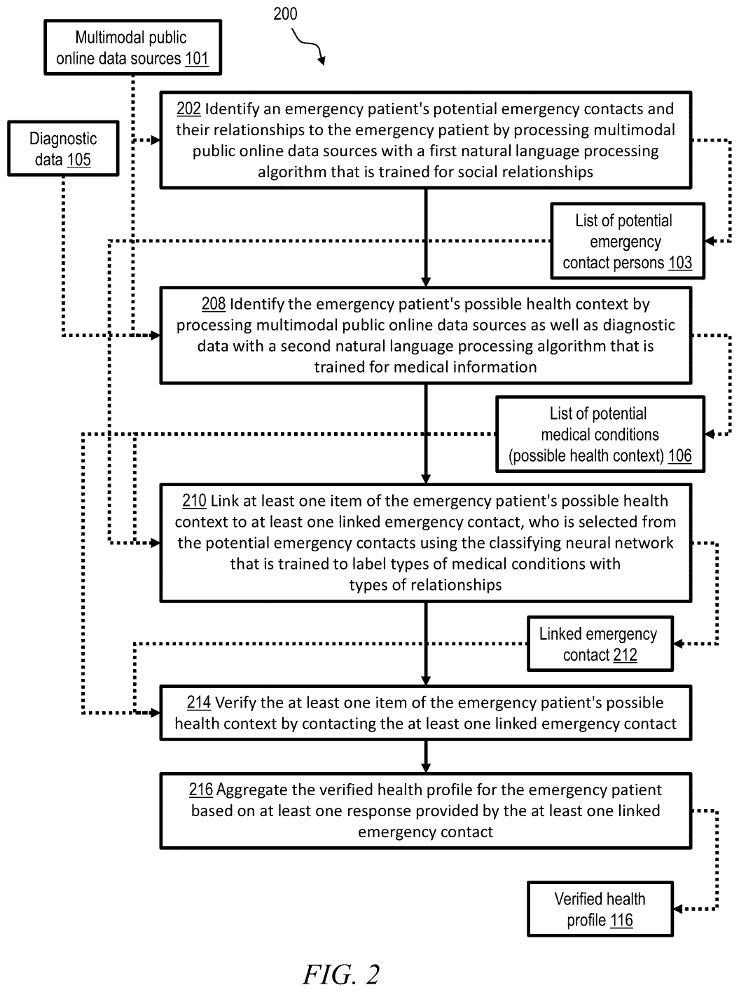

Thus, the system 100 implements a method 200 for aggregating verified health profiles in an emergency room, as depicted in a flowchart in FIG. 2. At 202, the first natural language processing module 102 identifies an emergency patient's potential emergency contacts 103 and their relationships to the emergency patient by processing multimodal public online data sources 101 with a first natural language processing algorithm that is trained for social relationships. At 208, the second natural language processing module 104 identifies the emergency patient's possible health context 106 by processing multimodal public online data sources 101 as well as diagnostic data 105 with a second natural language processing algorithm that is trained for medical information. At 210, the sorting module 110 links at least one item of the emergency patient's possible health context 106 to at least one linked emergency contact 212, who is selected from the potential emergency contacts 103 using, e.g., a classifying neural network that is trained to label types of medical conditions with types of relationships. At 214, the verifier 112 verifies the at least one item of the emergency patient's possible health context 106 by contacting the at least one linked emergency contact 212. Then at 216, the aggregator 114 aggregates the verified health profile 116 for the emergency patient based on at least one response provided by the at least one linked emergency contact.

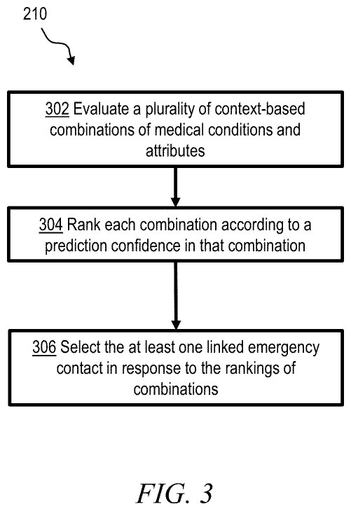

For example, as shown in FIG. 3, in one or more embodiments the step 210 of linking an item of health context 106 to a linked emergency contact 212 may include: at 302, evaluating a plurality of context-based combinations of medical conditions and attributes; at 304, ranking each combination according to a prediction confidence in that combination; and at 306, selecting the at least one linked emergency contact in response to the rankings of combinations.

By way of a non-limiting example of an implementation of the invention, suppose an individual who is involved in a road accident is brought to casualty. An exemplary system in accordance with aspects of the invention uses the patient's identification details obtained at the accident scene and the physical descriptions of the patient to find social media posts and profiles that match the patient. A non-intrusive blood pressure (BP) sensor takes measurements on the patient which are used to derive themes of hypertension and nutrition, since the reading was above threshold. These themes are further used to filter the results. When a positive identity is obtained, the system filters for connections of the identified person and further filters against connections who are also concerned about similar themes. The output is a possible next of kin. This information is summarized by the system and presented to the casualty doctors.

In another non-limiting example, an unknown and unconscious person is brought to casualty by an ambulance. The system uses the method in the preceding example above to identify the person. The system queries for all the person's media posts and uses it to deduce the person's non-clinical profile. The presence of posts about nutrition and exercise is used to inform the doctors at casualty to check if the person has a disease; e.g., diabetes or hypertension, and/or to be cautious while providing care methods that might contraindicate or interact with medications for these conditions.

In still another non-limiting example, a female person who was involved in a road accident is brought to the ER experiencing seizures. Phenytoin and Diazepam are currently available in the ER for treating seizures. The first responder's report includes the patient's identity (obtained from her ID), but no information on the next of kin or medical history. The system uses this information to search online sources and outputs fourteen potential emergency contact persons (PECPs). The system uses an ID-based open-source data query to finds a two-day old post in Facebook by the patient complaining of how “nausea and vomiting made my day the worst ever.” The system's health context filters/extracts concepts from the statement and identifies “nausea” and “vomiting” as possible symptoms of early stage pregnancy, food poisoning, ulcers, etc. The system's ranking algorithm ranks the PECPs and the patient's confidante scores highest based on the ‘early stages of pregnancy’ theme. The system contacts confidante who confirms that the patient is early in a pregnancy. This information is communicated to the medical emergency respondents, who rule out the use of Phenytoin in favor of Diazepam in treating the seizures as Phenytoin contraindicates with the pregnancy condition.

One or more embodiments are useful, for example, in the case of incapacitated patients. One or more embodiments advantageously employ a wide scope of open data that goes beyond patient records. One or more embodiments determine closeness of relationship with a patient based on social media networks and rank the potential emergency contact persons (PECPs) on each health condition identified based on relationship with the patient. One or more embodiments further include a notification engine to contact the identified PECPs. At least some embodiments identify the patient without using facial recognition.

One or more embodiments provides systems and methods for providing emergency personnel with non-documented health profile and contextual information about a patient whose medical history may not be immediately accessible; beyond what is available from conventional accessible information in a predefined medical history database. One or more embodiments provide techniques for deducing, contacting, and using the patient's next of kin to verify or provide additional contextual information about the emergency situation of a patient.

One or more embodiments generate a list of potential emergency contact persons (PECPs) for the purpose not only of notifying them but also of gaining more information regarding emergency victims. Information given by the PECPs as well as other existing information is aggregated to provide a helpful health profile of the individual that is enriched with contextual information. One or more embodiments employ techniques that utilize data from disparate sources, some of which contain unstructured data, and apply analytics to come up with useful information related to a patient's health.

Given the discussion thus far, and with reference to the accompanying drawings, it will be appreciated that, in general terms, an exemplary method, according to an aspect of the invention, includes at 202 identifying an emergency patient's potential emergency contacts and social relationships of the potential emergency contacts to the emergency patient by processing the emergency patient's Internet footprint with a first natural language processing algorithm that is trained for social relationships. Then, at step 208, identify the emergency patient's possible health context by processing at least one of the emergency patient's Internet footprint and diagnostic data with a second natural language processing algorithm that is trained for medical information using a generic classifier. At step 210, link at least one item of the emergency patient's possible health context to at least one linked emergency contact, who is selected from the potential emergency contacts using a sorting module 110 that is trained to associate types of medical conditions with attributes of emergency contacts. At step 214, verify the at least one item of the emergency patient's possible health context by contacting the at least one linked emergency contact. Then at step 216, aggregate a verified health profile for the emergency patient based on at least one response provided by the at least one linked emergency contact.

In one or more embodiments the method also includes, with the sorting module 110, evaluating, at step 302, a plurality of context-based combinations of medical conditions and attributes; ranking, at step 304, each combination according to a prediction confidence in that combination; and selecting, at step 306, the at least one linked emergency contact in response to the rankings of combinations.

In one or more embodiments the at least one linked emergency contact is contacted using an automated health condition inquiry.

In one or more embodiments the verified health profile is aggregated using speech-to-text technology and natural language processing to produce an electronic health record entry from an unstructured verbal response to the automated health condition inquiry.

In one or more embodiments the first natural language processing algorithm was trained (e.g. is pre-trained) on text corpora annotated with named entities. In one or more embodiments the second natural language processing algorithm was trained (e.g. is pre-trained) using domain specific data that includes at least one of a drug index, a drug bank, and FDA open source data.

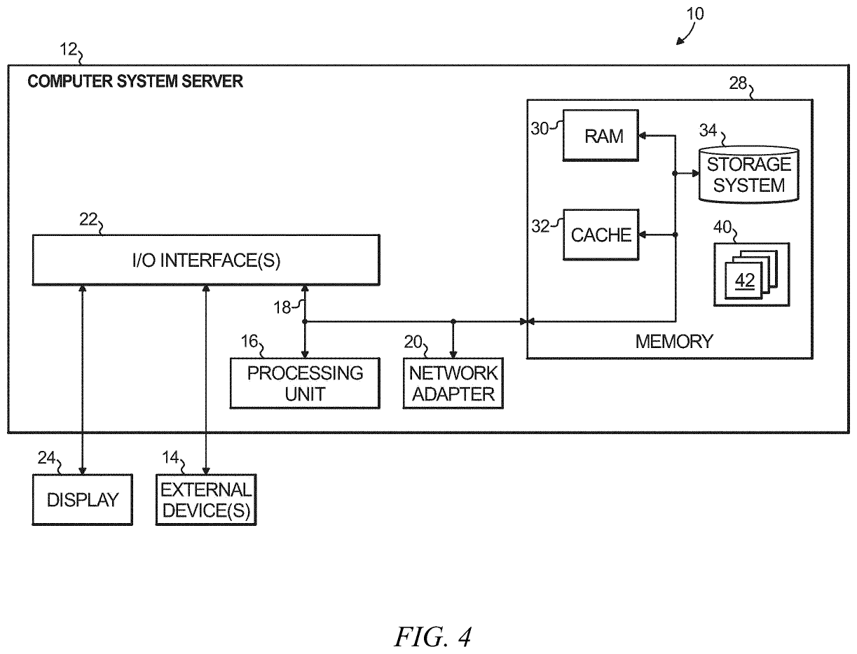

One or more embodiments of the invention, or elements thereof, can be implemented in the form of an apparatus including a memory and at least one processor that is coupled to the memory and operative to facilitate exemplary method steps, or in the form of a non-transitory computer readable medium embodying computer executable instructions which when executed by a computer cause the computer to facilitate exemplary method steps. FIG. 4 depicts a computer system that may be useful in implementing one or more aspects and/or elements of the invention. Referring now to FIG. 4, note a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network, and/or in conventional non-cloud environments. In a distributed cloud computing environment, program modules may be located in both local and remote computer system storage media including memory storage devices.

As shown in FIG. 4, computer system/server 12 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, and external disk drive arrays, RAID systems, tape drives, and data archival storage systems, etc.

Thus, one or more embodiments can make use of software running on a general purpose computer or workstation. With reference to FIG. 4, such an implementation might employ, for example, a processor 16, a memory 28, and an input/output interface 22 to a display 24 and external device(s) 14 such as a keyboard, a pointing device, or the like. The term “processor” as used herein is intended to include any processing device, such as, for example, one that includes a CPU (central processing unit) and/or other forms of processing circuitry. Further, the term “processor” may refer to more than one individual processor. The term “memory” is intended to include memory associated with a processor or CPU, such as, for example, RAM (random access memory) 30, ROM (read only memory), a fixed memory device (for example, hard drive 34), a removable memory device (for example, diskette), a flash memory and the like. In addition, the phrase “input/output interface” as used herein, is intended to contemplate an interface to, for example, one or more mechanisms for inputting data to the processing unit (for example, mouse), and one or more mechanisms for providing results associated with the processing unit (for example, printer). The processor 16, memory 28, and input/output interface 22 can be interconnected, for example, via bus 18 as part of a data processing unit 12. Suitable interconnections, for example via bus 18, can also be provided to a network interface 20, such as a network card, which can be provided to interface with a computer network, and to a media interface, such as a diskette or CD-ROM drive, which can be provided to interface with suitable media.

Accordingly, computer software including instructions or code for performing the methodologies of the invention, as described herein, may be stored in one or more of the associated memory devices (for example, ROM, fixed or removable memory) and, when ready to be utilized, loaded in part or in whole (for example, into RAM) and implemented by a CPU. Such software could include, but is not limited to, firmware, resident software, microcode, and the like.

A data processing system suitable for storing and/or executing program code will include at least one processor 16 coupled directly or indirectly to memory elements 28 through a system bus 18. The memory elements can include local memory employed during actual implementation of the program code, bulk storage, and cache memories 32 which provide temporary storage of at least some program code in order to reduce the number of times code must be retrieved from bulk storage during implementation.

Input/output or I/O devices (including but not limited to keyboards, displays, pointing devices, and the like) can be coupled to the system either directly or through intervening I/O controllers.

Network adapters 20 may also be coupled to the system to enable the data processing system to become coupled to other data processing systems or remote printers or storage devices through intervening private or public networks. Modems, cable modem and Ethernet cards are just a few of the currently available types of network adapters.

As used herein, including the claims, a “server” includes a physical data processing system (for example, system 12 as shown in FIG. 4) running a server program. It will be understood that such a physical server may or may not include a display and keyboard.

One or more embodiments can be at least partially implemented in the context of a cloud or virtual machine environment, although this is exemplary and non-limiting.

It should be noted that any of the methods described herein can include an additional step of providing a system comprising distinct software modules embodied on a computer readable storage medium; the modules can include, for example, any or all of the appropriate elements depicted in the block diagrams and/or described herein; by way of example and not limitation, any one, some or all of the modules/blocks and or sub-modules/sub-blocks described. The method steps can then be carried out using the distinct software modules and/or sub-modules of the system, as described above, executing on one or more hardware processors such as 16. Further, a computer program product can include a computer-readable storage medium with code adapted to be implemented to carry out one or more method steps described herein, including the provision of the system with the distinct software modules.

One example of user interface that could be employed in some cases is hypertext markup language (HTML) code served out by a server or the like, to a browser of a computing device of a user. The HTML is parsed by the browser on the user's computing device to create a graphical user interface (GUI).

Exemplary System and Article of Manufacture Details

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
identifying an emergency patient's potential emergency contacts and social relationships of the potential emergency contacts to the emergency patient by processing the emergency patient's Internet footprint with a first natural language processing algorithm that is trained for social relationships;
identifying the emergency patient's possible health context by processing at least one of the emergency patient's Internet footprint and diagnostic data with a second natural language processing algorithm that is trained for medical information using a generic classifier;
linking at least one item of the emergency patient's possible health context to at least one linked emergency contact, who is selected from the potential emergency contacts using a sorting module that is trained to associate types of medical conditions with attributes of emergency contacts;
verifying the at least one item of the emergency patient's possible health context by contacting the at least one linked emergency contact; and
aggregating a verified health profile for the emergency patient based on at least one response provided by the at least one linked emergency contact.

2. The method of claim 1, further comprising, with the sorting module, evaluating a plurality of context-based combinations of medical conditions and attributes, ranking each combination according to a prediction confidence in that combination, and selecting the at least one linked emergency contact in response to the rankings of combinations.

3. The method of claim 1 wherein the at least one linked emergency contact is contacted using an automated health condition inquiry.

4. The method of claim 3 wherein the verified health profile is aggregated using speech-to-text technology and natural language processing to produce an electronic health record entry from an unstructured verbal response to the automated health condition inquiry.

5. The method of claim 1 wherein the first natural language processing algorithm was trained on text corpora annotated with named entities.

6. The method of claim 1 wherein the second natural language processing algorithm was trained using domain specific data that includes at least one of a drug index, a drug bank, and FDA open source data.

7. A non-transitory computer readable medium embodying computer executable instructions which when executed by a computer cause the computer to facilitate a method of:
identifying an emergency patient's potential emergency contacts and social relationships of the potential emergency contacts to the emergency patient by processing the emergency patient's Internet footprint with a first natural language processing algorithm that is trained for social relationships;
identifying the emergency patient's possible health context by processing at least one of the emergency patient's Internet footprint and diagnostic data with a second natural language processing algorithm that is trained for medical information using a generic classifier;
linking at least one item of the emergency patient's possible health context to at least one linked emergency contact, who is selected from the potential emergency contacts using a sorting module that is trained to associate types of medical conditions with attributes of emergency contacts;
verifying the at least one item of the emergency patient's possible health context by contacting the at least one linked emergency contact; and
aggregating a verified health profile for the emergency patient based on at least one response provided by the at least one linked emergency contact.

8. The computer readable medium of claim 7, the method further comprising, with the sorting module, evaluating a plurality of context-based combinations of medical conditions and attributes, ranking each combination according to a prediction confidence in that combination, and selecting the at least one linked emergency contact in response to the rankings of combinations.

9. The computer readable medium of claim 7 wherein the at least one linked emergency contact is contacted using an automated health condition inquiry.

10. The computer readable medium of claim 9 wherein the verified health profile is aggregated using speech-to-text technology and natural language processing to produce an electronic health record entry from an unstructured verbal response to the automated health condition inquiry.

11. The computer readable medium of claim 7 wherein the first natural language processing algorithm was trained on text corpora annotated with named entities.

12. The computer readable medium of claim 7 wherein the second natural language processing algorithm was trained using domain specific data that includes at least one of a drug index, a drug bank, and FDA open source data.

13. An apparatus comprising:
a memory embodying computer executable instructions; and
at least one processor, coupled to the memory, and operative by the computer executable instructions to facilitate a method of:
identifying an emergency patient's potential emergency contacts and social relationships of the potential emergency contacts to the emergency patient by processing the emergency patient's Internet footprint with a first natural language processing algorithm that is trained for social relationships;
identifying the emergency patient's possible health context by processing at least one of the emergency patient's Internet footprint and diagnostic data with a second natural language processing algorithm that is trained for medical information using a generic classifier;
linking at least one item of the emergency patient's possible health context to at least one linked emergency contact, who is selected from the potential emergency contacts using a sorting module that is trained to associate types of medical conditions with attributes of emergency contacts;
verifying the at least one item of the emergency patient's possible health context by contacting the at least one linked emergency contact; and
aggregating a verified health profile for the emergency patient based on at least one response provided by the at least one linked emergency contact.

14. The apparatus of claim 13, the method further comprising, with the sorting module, evaluating a plurality of context-based combinations of medical conditions and attributes, ranking each combination according to a prediction confidence in that combination, and selecting the at least one linked emergency contact in response to the rankings of combinations.

15. The apparatus of claim 13 wherein the at least one linked emergency contact is contacted using an automated health condition inquiry.

16. The apparatus of claim 15 wherein the verified health profile is aggregated using speech-to-text technology and natural language processing to produce an electronic health record entry from an unstructured verbal response to the automated health condition inquiry.

17. The apparatus of claim 13 wherein the first natural language processing algorithm was trained on text corpora annotated with named entities.

18. The apparatus of claim 13 wherein the second natural language processing algorithm was trained using domain specific data that includes at least one of a drug index, a drug bank, and FDA open source data.
